(19) United States
(12) Patent Application Publication
(10) Pub. No.: US 2010/0280117 A1
(43) Pub. Date: Nov. 4, 2010

(54) Menorrhagia Instrument and Method for the Treatment of Menstrual Bleeding Disorders

(75) Inventors: Donald Patrick, Seattle, WA (US); Keith A. Moore, Loveland, OH (US); Brian J. Ruff, Cleves, OH (US); Ralph A. Heasley, Webster Groves, MO (US)

Correspondence Address: Davidson, Davidson & Kappel, LLC, 485 7th Avenue, 14th Floor, New York, NY, 10018, US

(73) Assignee: Xanodyne Pharmaceuticals, Inc., Newport, KY (US)

(21) Appl. No.: 12/770,185

(22) Filed: Apr. 29, 2010

Related U.S. Application Data
(60) Provisional application No. 61/174,362, filed on Apr. 30, 2009.

(51) Int. Cl. A61K 31/196; A61P 15/02

(52) U.S. Cl. 514/561

(57) ABSTRACT

Disclosed is a Menorrhagia Instrument and methods for the treatment of menstrual bleeding disorders.

This application claims priority under 35 U.S.C. §119(e) to U.S. Provisional Patent Application No. 61/174,362, filed Apr. 30, 2009, the disclosure of which is hereby incorporated by reference in its entirety.

### FIELD OF THE INVENTION

The invention is directed to a medical instrument designed for treatment of bleeding disorders (e.g., menorrhagia) in women. The instrument replaces the conventional heme based assay currently used to evaluate and to monitor bleeding conditions.

### BACKGROUND OF THE INVENTION

### Menstrual Bleeding

Menstrual Bleeding disorders encompass a number of conditions including bleeding associated with uterine fibroids, endometriosis, or bleeding as a result of deficiencies in the clotting process for example, von-Willebrand's disease. Studies suggest that as many as 11% of the women who experience heavy menstrual bleeding, suffer from an inherited bleeding disorder such as von Willebrand's disease. Excessive Menstrual Bleeding is menstruation at relatively regular intervals but with excessive blood loss over the menses period which may be prolonged. Heavy Menstrual Bleeding (also referred to as “Menorrhagia”) is a serious, persistent, and recurrent medical condition that is one of the most common complaints encountered by gynecologists and primary care physicians (Palep-Singh, 2007). A 2005 survey of 273 obstetrician/gynecologists found that they see an average of 18 to 25 symptomatic patients per month. Heavy Menstrual Bleeding is a hyperfibrinolytic condition defined as cyclic, normal intervals of menstruation with excessive volume. Menorrhagia is often associated with a disruption in daily routines, work, and sexual activity leading to a significant decrease in health-related quality of life and time lost from work or school. While Menorrhagia is rarely life threatening, when undiagnosed and untreated, it may over time cause iron deficiency anemia and increased fatigue, both of which affect normal life activities, relationships, social activities, and various aspects of mental well-being (irritation, anxiety). Left untreated it may be associated with subsequent morbidity including dysmenorrhea, hospitalization, red blood cell transfusions and chronic pain. Annually, approximately 10% of women of reproductive age report Menorrhagia (Rees 1991; van Eijkeren, 1992) and according to the Center for Disease Control (CDC), 3 million women of reproductive age report Menorrhagia yearly, 60% of which have no known etiology. Studies report that as many as thirty percent of premenopausal women perceive their menses to be excessive.

Women suffering from menorrhagia often have greater uterine fibrinolytic activity than women with normal cyclic menstrual blood loss (MBL). High concentrations of plasminogen activators are found in both the uterus and menstrual fluid (Albrechtsen, 1956a,b). Rybo (1966) found significantly higher concentration of endometrial plasminogen activators in women with excessive menstrual bleeding compared to women with normal menstrual loss.

Causes of Menorrhagia include pelvic diseases (myomata [fibroids], adenomyosis or uterine polyps), intrauterine contraceptive devices, and systemic disorders (coagulopathies such as thrombocytopenia or von Willebrand's disease, and hypothyroidism). In contrast to menorrhagia, the term ‘dysfunctional uterine bleeding’ refers to excessive, prolonged or irregular bleeding from the endometrium that is unrelated to systemic disease (Wathen, 1995), and is usually associated with anovulation. Menorrhagia is also distinguished from other ovulatory bleeding disorders, such as metrorrhagia (intermenstrual bleeding), menometrorrhagia (irregular heavy menstrual bleeding) and polymenorrhea (menstrual cycle less than 21 days).

### Diagnosis of Menstrual Blood Loss

In clinical trials, menstrual blood loss (MBL) is usually determined by measuring the amount of hemoglobin recovered from sanitary products during the menstrual cycle, using the alkaline hematin method (Fraser, 1994). However, it is important to remember that blood accounts for only about 50% of total menstrual flow, with endometrial transudate accounting for the remainder (Fraser, 1994). Total menstrual flow can be estimated by weighing of sanitary products or by comparisons with a pictorial blood loss assessment chart. However, the use of these quantitative and semi-quantitative methods is not practical in non-trial settings. Rather, the diagnosis of Menorrhagia in the healthcare clinic is made by medical providers on the basis of patient's perceived and self-reported medical history, routine laboratory assessments of the patient's general health status, and gynecological examinations.

Clinically heavy menstrual bleeding is sometimes defined as total blood loss exceeding about 80 ml per cycle or menses lasting longer than seven days. The volume lost however, varies widely. Clinically losses from about 30 ml to 60 ml, 60 to 80 ml, 80 to 100 ml, to as high as 1000 ml per cycle are observed. Menstrual blood losses of 50 to 60 ml are associated with a negative iron balance and iron deficiency anemia is diagnosed in about 67% of the women who lose an excess of 80 ml per day. Other criteria for diagnosing the condition include measuring the number and size of blood clots in the meneges, or monitoring the use of pads or tampons. It is estimated that perhaps only ten percent of women who perceive their loss to be excessive actually fall within the clinical definition. The 80 ml definition has been repeatedly questioned, and alternative definitions broadened the blood loss range used for patient evaluations.

Blood loss volume assessments commonly require the collection and preservation of menstrual pads or tampons, the extraction of the pads and the accurate measurement of the blood content. Women are instructed to collect all sanitary towels and tampons during the course of the menstrual diagnosis period or the course of a clinical study period. Blood loss can be measured by extraction of the blood from the sanitary material with 5% sodium hydroxide followed with a spectrophotometric measurement of hematin at a wavelength of about 540 nm. The total blood loss can be calculated for an individual by comparison of the patients plasma blood hemoglobin measurement with the collected hemoglobin values.

The collection of the blood sample discourages the routine use of the test in the diagnosis or in the treatment of the condition. In the course of a routine visit with a physician other blood work may be appropriate but lacks a casual relation to the heavy bleeding disorder. The battery of routine laboratory tests may include patient blood hemoglobin, haematocrit, platelet count, bilirubin, serum creatinine and serum ferritin. In sum, diagnosis in the routine course of practice relies heavily on the woman's perception of the volume of blood lost during menses.

### Diagnosis and Treatment of Heavy Menstrual Bleeding Disorders (Menorrhagia)

A number of medical and surgical interventions are available to treat menstrual bleeding disorders. Currently available non-surgical treatments for heavy bleeding disorders, include, hormonal treatments (e.g., oral contraceptives), high-dose progestin therapy, desmopressin acetate, ethamsylate, nonsteroidal anti-inflammatory drugs (NSAIDs), the antifibrinolytic drugs aminocaproic acid and tranexamic acid. Even with the drug treatments available, surgery remains a common treatment.

Although not approved for menorrhagia in the US, use of oral contraceptives for menorrhagia is widely accepted. Oral contraceptives may not be a preferred therapy for some women because of age (younger females), unwanted side effects (nausea and vomiting, breakthrough bleeding, weight change, migraines and depression), and safety concerns (increased risk of thromboembolism, stroke, myocardial infarction, hepatic neoplasia and gall bladder disease). High-dose progestin (synthetic versions of the hormone progesterone) may also be given to women with menorrhagia, either orally or by a progestin-releasing device inserted into the uterus (intrauterine device). Side effects include nausea, bloating, mood changes, and breast tenderness.

Although it is typically a last resort, desmopressin acetate is sometimes used to help lighten menstrual flow in women with menorrhagia. The effectiveness of desmopressin is thought to vary between individuals. Side effects include headache, tachycardia, facial flushing, and rare reports of thromboembolism.

NSAIDs are sometimes used to treat menorrhagia as they may reduce blood flow while providing analgesia for pain associated with the condition (Shaw, 1994). Side effects associated with chronic NSAID use include gastrointestinal bleeding, ulceration, and perforation; and renal effects such as hyperkalemia, hyponatremia, acute renal insufficiency, interstitial nephritis, and renal papillary necrosis.

Hysterectomy or endometrial resection are options if other forms of therapy are not effective or are unsuitable for some reason. Possible surgical complications include infection, uterine perforation, and other complications associated with major surgery.

Antifibrinolytic drugs, such as s-aminocaproic acid and tranexamic acid (immediate-release formulation) have been used to treat HMB in women with or without a diagnosed bleeding disorder (van Eijkeren, 1992; Bonnar, 1996; Vermylen, 1968; Nilsson, 1965). The available evidence from published literature suggests that tranexamic acid at doses of ˜4 g/day (typically 1 g every 6 hours) is effective in the treatment of HMB and is associated with few side effects (Callender, 1970; Dunn, 1999; Edlund, 1995; Preston, 1995). In Sweden, the average dose of tranexamic acid to treat HMB is 3.9 g/day (Rybo, 1991). Thus, tranexamic acid is used extensively in Europe, Canada, Asia, Japan, Australia and New Zealand to treat menorrhagia, but is not approved for this indication in the US.

Tranexamic acid is a competitive inhibitor of plasminogen activation (see review by Dunn, 1999). Binding of tranexamic acid to plasminogen does not prevent conversion of plasminogen to plasmin by tissue plasminogen activator, but the resulting plasmin/tranexamic acid complex is unable to bind to fibrin. Thus, enzymatic breakdown of fibrin by plasmin (fibrinolysis) is inhibited. At higher concentrations, tranexamic acid is also a noncompetitive inhibitor of plasmin.

Before medical and surgical interventions can be initiated, diagnosis of a heavy menstrual bleeding disorder must be accomplished.

Diagnosis and treatment of disease often depends on the patient's perception and subsequent description of symptoms, the physician's evaluation of the patient's description, the physician observations of the patient and laboratory test results. Menstrual bleeding disorders do not lend themselves to physician observation or to routine laboratory testing. Patient observations and the physician's evaluation of the patient's description are subjective and thus variable. In addition a women's medical history has been found to be a poor predictor of menstrual blood loss. Neither the duration of menses nor the number of sanitary pads worn accurately corresponds to the woman's actual menstrual blood loss (Chimbira, Haynes, year). An objective assessment of blood loss using the alkaline haematin assay has been shown to be reproducible but it is not suited for routine clinical use by healthcare providers. To date no effective instrument for reliably diagnosing and/or monitoring the treatment of menstrual bleeding disorders has been developed despite the significant number of women who suffer from these conditions.

Previously, studies have focused on the impact of symptoms of bleeding disorders on patients' health related quality of life. As the effects of menstrual bleeding disorders are primarily symptomatic, the subjective outcome namely symptom alleviation, cannot be objectively measured. In research from European countries where the antifibrinolytic drug tranexamic acid is currently available, treatment with this antifibrinolytic has reduced heavy menstrual bleeding by 40-50% and improved the health-related quality of life of affected women on measures of social activity, work performance, productivity, cleanliness, overall functioning and tiredness.

Jenkinson et al, Quality in Health Care 1996; 5; 9-12 evaluated the validity and internal reliability of the short form-36 (SF36) health survey questionnaire in women presenting with menorrhagia. The study concluded that several questions on the questionnaire were difficult to answer for patients with heavy menstrual bleeding. Such problems were suggested as possible interferences with the validity of the measure. Jenkinson warns that because a subjective measure works well in one population or with one group, this cannot be taken to imply its appropriateness for all groups or conditions.

Edlund, in an abstract from a seminar on Dysfunctional Uterine Bleeding, Feb. 23, 1994, indicates that a questionnaire was used in a Swedish study of 2205 women who described their menstruation as excessive.

Winkler in a study based in part on the Edlund work, concluded that the treatment of heavy menstrual bleeding with tranexamic acid increased the quality of life of the treated patients. The Winkler study was an open label uncontrolled usage study which included 849 patients. A questionnaire was used prior to treatment and after the first and third menstruation. The study indicates that 80% of the women were satisfied with the treatment. The questionnaire used a series of eight question combined with an assessment by the patients of the change in quantity of menstrual flow.

Ruta, D. A., Quality of Life Research, 4, (33-40), 1995 finds that menorrhagia is a common problem in gynecological practice and that women seek professional help primarily because of the deleterious effect on their quality of life. Ruta recognizing the importance of evaluating the effectiveness of the treatments developed a questionnaire based on the type of questions frequently asked when taking a gynecological history. A series of questions were devised which assessed fifteen factors including the duration of the period, the regularity of the period, pain, problems with soiling/staining, interference with work, interference with leisure. Ruta concluded that the clinical questionnaire may be useful in selecting patients for hysterectomy and assessing the outcome of conservative treatment especially in combination with the SF-36 questionnaire.

### Diagnostic Test for Menstrual Bleeding

The alkaline haematine test described above provides quantitative assessments of the extent of menstrual bleeding. This test allows the physician to diagnose and monitor the progress of a women's menstrual process. However the test is impractical and difficult to perform. The test requires women to capture used menstrual pads over the course of her period, preserve the samples in a condition such that the blood content within the pad may be accurately extracted and quantitated. Requesting a patient to perform menses sample collection may be practical in the course of a clinical trial where procedures are specified and monitored however, in routine medical practice, the use of such a test procedure to diagnose and monitor a women's menstrual bleeding is impractical and the data generated is unreliable.

The need remains to develop an assessment system which replaces previously studied diagnostic techniques and the alkaline haematine test and provides a reliable measure of both the occurrence of the disorder and the progress of the disorder. The present invention fills this need by providing a Heavy Menstrual Bleeding Instrument (HMBI) which is capable of diagnosing, and monitoring the treatment of a patient with a menstrual bleeding disorder.

There also remains a need to provide Heavy Menstrual Bleeding (HMB) therapy that is safe, efficacious and only administered during the monthly period of heavy menstruation, addresses the excessive fibrinolysis implicated in many causes of menorrhagia, and fills a currently recognized unmet medical need in the US. Therapy for HMB is expected to reduce the incidence and extent of iron-deficiency anemia, and to provide a nonhormonal medical therapy option in lieu of the numerous invasive procedures (e.g., transcervical endometrial resection) and major surgery (hysterectomy) performed annually.

### OBJECTS AND SUMMARY OF THE INVENTION

An object of this invention is to provide methods for diagnosing and treating woman of reproductive age with heavy menstrual bleeding as a result of cyclic ovarian activity as the major complaint. This may be in association with uterine fibroids or dysfunctional uterine bleeding in the absence of visible pathology. The women are, or may be in need of antifibrinolytic therapy. The women suffering from heavy menstrual bleeding and in need of diagnosis and possible antifibrinolytic therapy can be of any age. For example, the age of the women may range from about 10 to about 14, from about 12 to about 18, from about 14 to about 30, from about 16 to about 58 years of age. The women contemplated also include those women between the ages of from about 20 to about 39, from about 28 to about 48, from about 40 to about 55 and from about 50 to about 58.

It is an objective of this invention to provide an interactive instrument for diagnosing and monitoring excessive menstrual bleeding in patients in need of antifibrinolytic therapy based on patient derived observations.

It is an object of this invention to provide an instrument which detects heavy menstrual blood loss in patients in need of antifibrinolytic therapy.

It is an object of this invention to provide an instrument which identifies patients with heavy menstrual blood loss who are in need of antifibrinolytic therapy.

It is an object of this invention to provide an instrument that monitors the decrease in heavy menstrual blood loss for patients during the course of antifibrinolytic therapy.

It is an object of this invention to provide a validated interactive instrument for excessive menstrual bleeding based on patient derived observations to diagnose and to monitor patient response to antifibrinolytic therapy.

It is an object of certain embodiments of the invention to provide an instrument for assessing heavy menstrual bleeding in which a patient is losing from about 30 to about 600 ml of blood per cycle, from about 40 to 500 ml of blood, from about 50 to 400 ml of blood per cycle, about 60 to 200 ml of blood, from about 75 to about 250 ml of blood per cycle.

It is an object of certain embodiments of the invention to provide an instrument for monitoring heavy menstrual bleeding in which a patient perceived blood loss is heavy or very heavy. The loss as measured by the alkaline hematin test is from about 30 to about 1000 ml of blood per cycle of blood per cycle, from about 40 to 800 ml of blood, from about 50 to 400 ml of blood per cycle, about 60 to 200 ml of blood, from about 75 to about 250 ml of blood per cycle from 100 to about 300 ml of blood from about 115 to about 650 ml of blood per cycle and is prescribed an antifibrinolytic agent.

In accordance with the above mentioned objects, the present invention is directed to a method of treating heavy menstrual bleeding. In certain embodiments the method employs measures which quantify a patient's most recent menstrual period. Such measures include, for example: a) determining a patient's perceived quantity of blood loss during their most recent menstrual period; b) determining how much the patient's perceived blood loss limited their work outside and inside the home; c) determining how much the patient's perceived blood loss limited their physical activities; d) determining how much the patient's perceived blood loss limited their social and leisure activities; e) determining the specific activities that were limited by the patient's perceived blood loss.

Once one or all of the measures have been compiled, a patient for treatment of heavy menstrual bleeding is identified based at least on the quantity of blood loss perceived by that patient as at least heavy and optionally an impairment of at least one function. A therapeutically effective treatment regimen of an antifibrolytic such as tranexamic acid is administered to the patient in need of treatment, wherein the treatment regimen is administered for at least the first day of menstruation, for at least the first two days of menstruation, for at least the first three days of menstruation, for the duration of menstruation.

The present invention is also directed to methods of evaluating the effectiveness of a treatment regimen administered for heavy menstrual bleeding. The evaluation compares the patients most recent menstrual period with a previous period fro which comparative data are available. The comparative data used for the evaluation include, for example: a) determining a patient's perceived blood loss during their most recent menstrual period; b) determining how much the patient's perceived blood loss limited their work outside and inside the home; c) determining how much the patient's perceived blood loss limited their physical activities; d) determining how much the patient's perceived blood loss limited their social and leisure activities; e) determining the specific activities that were limited by the patient's blood loss; and f) determining the patient's perceived blood loss during the most recent menstrual period in comparison to the blood loss during the patient's previous menstrual period, g). determining the patient's change in measures of work limitation, measures of physical limitation, measures of social/leisure limitation, during the most recent menstrual period in comparison to the patient's previous menstrual period.

In accordance with the above-mentioned objects, the method of the present invention is directed to a method of using tranexamic acid in the treatment of heavy menstrual bleeding for a female patient in need thereof comprising: a) determining the perceived amount of blood loss during a patient's most recent menstrual period, wherein the perceived amount of blood loss is determined based upon the patient's response about their blood loss on a four point scale; said scale comprising a response selected from the group consisting of i) “light”, ii) “moderate”, iii) “heavy”, and iv) “very heavy”; b) identifying a patient in need of therapy as one with a perceived blood loss of greater than or equal to a response of “heavy”; c) administering a therapeutically effective treatment regimen of a modified release tranexamic acid formulation for treating heavy menstrual bleeding, said treatment regimen to be administered beginning at the start of the patient's next menstrual period for at least about 3 days; d) determining a change in the perceived amount of blood loss after administration of the tranexamic acid treatment regimen, and determining whether that change was meaningful to the patient, wherein the change in the perceived amount of blood loss is determined based upon the patient's response on a three point scale comprising a response selected from the group consisting of i) “about the same”, ii) “better”, and iii) “worse”, and the meaningfulness of the change to the patient is based upon the patient's response about the change on a two point scale comprising a response selected from the group consisting of “yes” and “no”; and e) classifying the patient as a responder when the patient's response to the change in perceived amount of blood loss in step (d) is “better”, and the patient perceives the change to be meaningful.

For purposes of the present invention, the following definitions are provided:

“Therapy” for excessive menstrual bleeding is defined for the purpose of this invention as one or more courses of treatment with an antifibrinolytic agent such as, but not limited to, tranexamic acid, aminocaproic acid, and any pharmaceutically acceptable salts, esters, derivatives, pro-drugs, metabolites, and analogues of any of the foregoing antifibrinolytic agents.

The term “heavy menstrual bleeding” is defined for purposes of the present invention as a perceived blood loss of at least heavy to very heavy which may correspond to a periodic blood loss of at least about 30 ml per cycle to as much as 1000 ml per cycle as measured by the alkaline hematin test. The periodic blood loss perceived or as measured with the alkaline hematin test may vary depending on the severity of the condition and the physiological make up of the individual patient. Therefore, heavy menstrual bleeding may include periodic blood losses of at least about 30 ml per cycle. Losses from between about 30 ml, about 40 ml, about 50 ml, about 60 ml, about 70 ml, about 80 ml, about 90 ml to about 300 ml are contemplated as are losses greater than 300 ml, such as for example, losses between about 300 ml to about 1000 ml.

### BRIEF DESCRIPTION OF THE DRAWINGS

FIG. 1 is a listing of the Menorrhagia Impact Measures of the present invention.

FIG. 2 is a graph of Menorrhagia Instrument measure #1 percentage of patients and normals indicating each response at baseline (BL) and at one (1) month (M1).

FIG. 3 is a graph of the limitations of social and leisure activities (LSLA) in women with Heavy Menstrual Bleeding (HMB) in accordance with the treatment regimens administered in Examples VII and VIII.

FIG. 4 is a graph of the mean menstrual blood loss change from the clinical studies of Examples VII and VIII.

### DETAILED DESCRIPTION

Applicant conducted studies of the safety and efficacy of the antifibrinolytic tranexamic acid. As part of these studies a diagnosis and treatment instrument was designed. The instrument reliably identifies and monitors heavy menstrual bleeding patients and can be used in conjunction with an antifibrinolytic agent to diagnose and monitor the treatment of heavy menstrual bleeding.

A Heavy Menstrual Bleeding Instrument (HMBI) of the invention reliably captures the diagnosis and treatment of the disease by measuring the impact of treatment on the symptoms associated with heavy menstrual bleeding. The information obtained from individual patient responses to the measures described in the methods of the present invention correlates to blood loss as measured by the alkaline hematin test. For example, data from the measures of social, leisure and/or physical activity symptoms, correlate with the volume of blood loss, and the change in the intensity of these symptoms correlates with the change in volume of blood lost, thus providing a measurement for the successful diagnosis and evaluation of treatment of bleeding disorders.

The instrument of the present invention measures specific aspects of the patient's monthly menstrual period. The measures correlate with the diagnosis of heavy menstrual bleeding and with the course of antifibrinolytic treatment. Further each of the measures individually correlate with quantity of blood loss as measured by the alkaline Hematin test. The symptomatic measures include: 1) a functional assessment measure; and ii) a pharmacology (or therapy assessment) measure.

The functional assessment measure of symptoms is further factored into segments which include 1) a measure of functional impairment generally; 2) impairment of necessary activities; and 3) impairment of discretionary activities.

The pharmacology domain provides an assessment of the severity of the menstrual period.

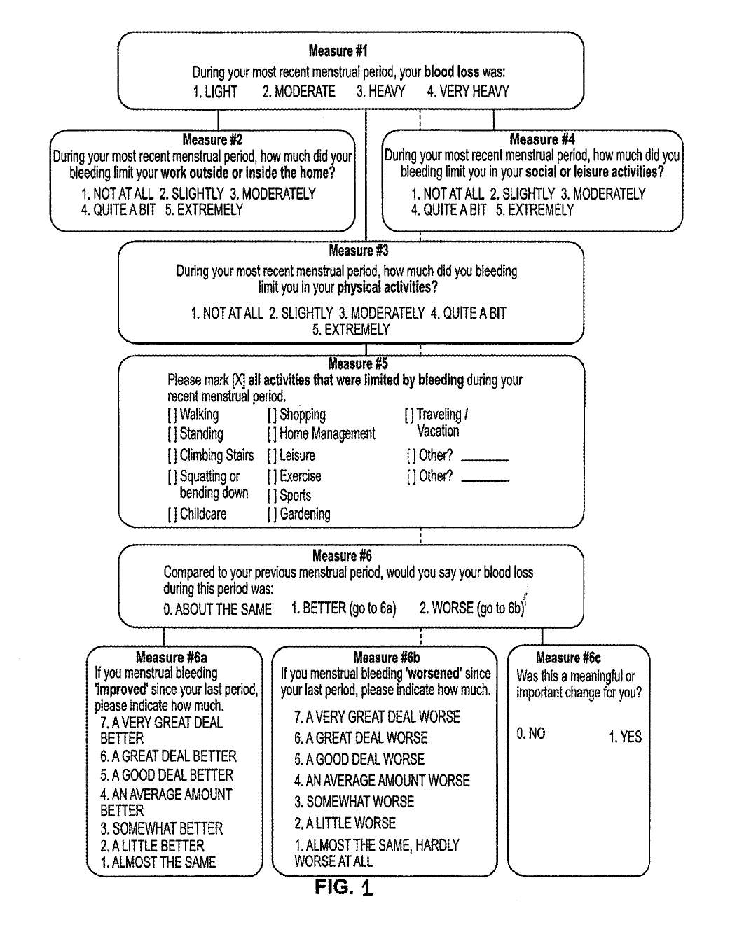

Specific symptomatic measures may be directed to an initial patient assessment and to the treatment period (pharmacology measure). Examples of specific measures would include examples of initial patient assessment measures (measures 1-4 listed in the Menorrhagia Instrument of FIG. 1); and therapy assessment measures (measures 1-4 together with measures 6, 6a, 6b and 6c contained in the Menorrhagia Instrument of FIG. 1).

In certain embodiments, the present invention is directed to a method of diagnosing and treating heavy menstrual bleeding, wherein the initial diagnoses of heavy menstrual bleeding is accomplished by evaluation of the most recent menstrual period on the basis of one, some or all of the prescribed symptomatic measures of FIG. 1. Measures which may be used as part of the initial patient assessment include, for example: a) determining a patient's perceived blood loss during their most recent menstrual period; b) determining how much the patient's blood loss limited their work outside and inside the home; c) determining how much the patient's blood loss limited their physical activities; d) determining how much the patient's blood loss limited their social and leisure activities; and e) determining the specific activities that were limited by the patient's blood loss.

The assessment of the patient's perceived blood loss during their most recent menstrual period may include an inquiry such as “during your most recent menstrual period, your blood loss was”. The assessment may then quantify the patient response as a blood loss that was: i) light, ii) moderate, iii) heavy, or iv) very heavy. Alternatively, the measure may be quantified in terms of a scale of from one to four where one represents light, two represents moderate, three represents heavy and four represents very heavy.

The assessment of a patient's limitation due to the blood loss may include and evaluation of the patient's blood loss limitation on physical activities and/or how much the patient's blood loss limited their social and leisure activities. Assessment of the limitations on work, physical, social and leisure activities may be quantitated as: i) not at all, ii) slightly, iii) moderately, iv) quite a bit, or v) extremely. Alternatively the measure may be quantified in terms of a scale of from one to five where one represents not at all, two represents slightly, three represents moderately, four represents quite a bit, and five represents extremely.

Activities limited may include, but are not limited to, walking, standing, climbing stairs, squatting or bending down, playing with children and attending school activities. Home management activities include, but are not limited to, cooking, cleaning, yard work, and laundry. Leisure activities may include, but are not limited to, dancing, dinner, and movies. Sports activities may include, but are not limited to, tennis, golf, running, swimming, hiking, biking, boating, baseball, softball, basketball, soccer, fencing, volleyball, and other sports related activities.

Once the initial patient assessment measures have been completed and the patient has been identified as in need of treatment, the patient is administered a therapeutically effective treatment regimen of an antifibrinolytic agent. Suitable antifibrinolytic agents contemplated for use in the present invention include, but are not limited to tranexamic acid, aminocaproic acid, pharmaceutically acceptable salts, esters, derivatives, pro-drugs, metabolites, and analogues of any of the foregoing antifibrinolytic agents.

In certain embodiments the preferred antifibrinolytic agent is tranexamic acid. The tranexamic acid utilized in the present invention can be formulated into any suitable dosage form. Preferably, the tranexamic acid is in the form of a release modified tranexamic acid formulation.

When the preferred antifibrinolytic is tranexamic acid, the therapeutically effective treatment regimen contemplated by the present invention includes administration of a single dose of a tranexamic acid ranging from about 650 mg to about 1300 mg three (3) times a day for at least one day of menstruation, but not more than five days (or 15 single doses). The treatment regimen may be administered for at least one day; for at least the first two days, for at least the first three days, for days two through three, for days two to three, for the duration of menstruation.

In certain embodiments the tranexamic acid treatment regimen for treating the heavy menstrual bleeding includes administration of a single dose of about 650 mg to about 1.3 gm of a modified release formulation three (3) times a day, wherein the modified release formulation contains the tranexamic acid in combination with a modified release material

In certain other embodiments, the present invention is directed to a method of evaluating the effectiveness of a treatment regimen administered for heavy menstrual bleeding.

Evaluation of the effectiveness of the treatment regimen can be initiated at the end of the patient's menstrual period, but prior to completion of the menstrual cycle. The post-menstruation measures provide in part the pharmacology (or therapy assessment) measure described above.

The pharmacology assessment may begin with one or more of the same series of measures utilized during the initial patient assessment, which include: a) determining a patient's perceived blood loss volume during their most recent menstrual period; b) determining how much the patient's blood loss limited their work outside and inside the home; c) determining how much the patient's blood loss limited their physical activities; d) determining how much the patient's blood loss limited their social and leisure activities; e) determining the specific activities that were limited by the patient's blood loss.

Alternatively, an evaluation of the effectiveness of the treatment regimen may require determining the change in the patient's perceived blood loss during the most recent menstrual period in comparison to the blood loss during the patient's previous menstrual period, measure 1 of FIG. 1 and/or an assessment of the improvement achieved, measure 6 of FIG. 1.

For example, a change in the patients perceived blood loss of about one unit for example from “heavy” to “moderate” or from a score of 3 (“heavy”) to a score of 2 (“moderate”) would provide the basis for continued treatment. While a perceived loss of less than one unit would suggest either a discontinuation of treatment or a second course after which the evaluation would be reconsidered. Alternatively, or in addition to the blood loss assessment, the practitioner may rely on the assessment in which the comparison of perceived loss is assessed as: i) “about the same”, ii) “better”, and iii) “worse”, as prescribed in measure 6 in FIG. 1. When a patient's response is “about the same”, an alternative treatment regimen may be considered for the next menstrual period. The practitioner may also reconsider re-administering the same treatment regimen for an additional menstrual period and later re-evaluate. When a patient's response is “better”, the assessment may continue by requiring the patient to provide further information about the improvement in menstrual bleeding. For example, the assessment may include “if your menstrual bleeding improved since your last period, please indicate how much” (measure 6b of the MI of FIG. 1). Answers to this inquiry about an improvement in menstrual bleeding may require the patient to provide an answer such as: i) a very great deal better; ii) a great deal better; iii) a good deal better; iv) an average amount better; v) somewhat better; vi) a little better; or vii) almost the same, hardly better at all. Alternatively the answers can be scaled on a seven unit scale where “a very great deal better” is assigned a value of 7 and “almost the same” is valued as 1.

When a patient's response to measure 6 is “worse”, the inquiry continues by requiring the patient to provide further data characterizing the change in menstrual bleeding. For example, the inquiry may determine “if your menstrual period worsened since your last period, please indicate how much” (measure 6c of MI of FIG. 1). Data for this measure to a worsening in menstrual bleeding may require the patient to provide a ranking such as: i) “a very great deal worse”; ii) “a great deal worse”; iii) “a good deal worse”; iv) “an average amount worse”; v) “somewhat worse”; vi) “a little worse”; or vii) “almost the same, hardly worse at all”. As before the answers may be scaled on a seven unit scale where-1 is “almost the same” and -7 is “a very great deal worse”.

The comparison of perceived blood loss which results in an improvement of at least one unit as measured by measure 1 of FIG. 1 and/or an assessment of a perceived blood loss which is “better” as provided in measure six of FIG. 1 may proceed by assessing whether the improvement “was a meaningful or an important change” to the patient (measure 6c of MI of FIG. 1).

The information obtained about the “improvement” or “worsening” in menstrual bleeding allows the practitioner to make an evaluation of the effectiveness of the treatment regimen which correlates with the change in blood loss as measured by the alkaline hematin test and demonstrated with clinical trial data.

The method for evaluating the effectiveness of a treatment regimen of the present invention may be repeated after each menstrual period. The data obtained from the initial patient assessment and the subsequent pharmacology (therapy assessment) can be stored into a computer database and utilized for future diagnostic and/or evaluation purposes.

In certain other embodiments, the present invention is directed to a method of treating heavy menstrual bleeding. The method involving, evaluating symptomatic data gathered from the measures individually or collectively as described in FIG. 1. (items one through four and six as discussed above) to determine the need for therapy and then administering, to a patient in need, a therapeutically effective treatment regimen of an antifibrinolytic agent, e.g., a release modified tranexamic acid formulation, wherein the treatment regimen is to be administered for part or for the duration of menstruation, but no longer than 5 days during the patient's menstrual cycle.

The present invention is further described with regard to the following examples.

### DETAILED DESCRIPTION OF THE PREFERRED EMBODIMENTS

In clinical trials the primary goal is to obtain definitive evidence regarding the benefit to risk profile of the pharmacotherapy. One of the most challenging design tasks in studies of heavy menstrual bleeding which is a subjective complaint is the choice of efficacy endpoints or outcome measures. The Applicants have established two criteria for assessing the clinical relevance of the reduction in menstrual blood loss in the clinical efficacy studies. The first criterion was that the mean reduction in menstrual blood loss should be greater than 50 mL. The second criterion was based on the correlation between the reduction in menstrual blood loss and the subjects' perception of a meaningful symptomatic change, derived from blinded data from the measures of the Menorrhagia Instrument (MI) in the first treated menstrual period in the menstrual cycle during a controlled clinical study for safety and efficacy of tranexamic acid in heavy menstrual Bleeding. Analysis of the data for the symptomatic measures of the Menorrhagia Instrument (MI, measure six, FIG. 1) established that a menstrual blood loss reduction of at least 36 mL as defined by the alkaline hematin test was regarded as meaningful by the clinical patients. The mean reduction in menstrual blood loss in patients treated with a tranexamic acid formulation at 1.9 and at 3.9 g/day met both criteria for a clinically meaningful result. Data from Menorrhagia Instrument (MI, measure six, FIG. 1, which establishes that the treatment was meaningful to the patient provides the treating practitioner with an assessment of patient response to tranexamic acid therapy.

### Example I

Modified release 650 mg tranexamic acid tablets were prepared and coated with film coating. The ingredients are listed in Table 1 below:

TABLE 1

| | Quantity | Quantity |
|---|---|---|
| | per batch | per tablet |
| Ingredient | (kg) | (mg) |
| Active Ingredient | | |
| Tranexamic Acid, EP | 84.50 | 650.0 |
| Inactive Ingredients | | |
| Microcrystalline Cellulose NF (Avicel PH 101) | 5.753 | 44.25 |
| Colloidal Silicon Dioxide NF | 0.0975 | 0.75 |
| Pregelatinized Corn Starch, NF | 6.435 | 49.50 |
| Hypromellose, USP (Methocel K3 Premium LV) | 19.110 | 147.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water USP* | 17.550 | 135.00 |
| Film Coating (Inactive Ingredients)** | | |
| Opadry White YS-1-7003 | 4.305 | — |
| Purified Water, USP | 38.750 | — |

*Purified water is removed during processing
**6 kg excess prepared to account for losses during transfer

The formulation of Example I was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the hypromellose USP Methocel K3 Premium to the V-blender. Blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets to desired weight.
13. After compression, spray coat the compressed dosage forms with the Opadry White in water.

### Example II

In example 2, modified release 650 mg tranexamic acid tablets were prepared as in Example I, without the film coating, having the ingredients listed in the Table 2 below:

TABLE 2

| | Quantity | Quantity |
|---|---|---|
| | per batch | per tablet |
| Ingredient | (kg) | (mg) |
| Active Ingredient | | |
| Tranexamic Acid, EP | 84.50 | 650.0 |
| Inactive Ingredients | | |
| Microcrystalline Cellulose NF (Avicel PH 101) | 5.753 | 44.25 |
| Colloidal Silicon Dioxide NF | 0.0975 | 0.75 |
| Pregelatinized Corn Starch, NF | 6.435 | 49.50 |
| Hypromellose, USP (Methocel K3 Premium LV) | 19.110 | 147.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water USP* | 17.550 | 135.00 |

*Purified water is removed during processing

The formulation of Example II was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the hypromellose USP Methocel K3 Premium to the V-blender. Blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets to desired weight.

### EXAMPLES III A-B

In Example IIIA, immediate release 650 mg tranexamic acid tablets were prepared having the ingredients listed in Table 3 below:

TABLE 3

| | Quantity | Quantity |
|---|---|---|
| | per batch | per tablet |
| Ingredient | (kg) | (mg) |
| Active Ingredient | | |
| Tranexamic Acid, EP (650 mg/tab) | 84.50 | 650.0 |
| Inactive Ingredients | | |
| Microcrystalline Cellulose, NF (Avicel PH 101) | 5.753 | 44.25 |
| Microcrystalline Cellulose, NF (Avicel PH 102) | 10.660 | 82.00 |
| Colloidal Silicon Dioxide, NF | 0.0975 | 0.75 |
| Pregelatinized Corn Starch, NF | 6.435 | 49.50 |
| Croscarmellose Sodium, NF | 19.50 | 15.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water, USP* | 17.550 | 135.00 |
| Film Coating (Inactive Ingredients)** | | |
| Opadry White YS-1-7003 | 4.110 | — |
| Purified Water, USP | 36.990 | — |

*Purified water is removed during processing
**6 kg excess prepared to account for losses during transfer

The formulation of Example IIIA was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized cornstarch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the croscarmellose sodium and MCC to the V-Blender and blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets.
13. After compression, spray coats the compressed dosage forms with the Opadry White in water.

In Example IIIB, 650 mg delayed release tranexamic acid tablets were prepared having the ingredients listed in Table 4 below:

TABLE 4

| | Quantity | Quantity |
|---|---|---|
| | per batch | per tablet |
| Ingredient | (kg) | (mg) |
| Active Ingredient | | |
| Tranexamic Acid, EP | 84.50 | 650.0 |
| Inactive Ingredients | | |
| Microcrystalline Cellulose, NF (Avicel | 5.753 | 44.25 |
| PH 101) | | |
| Microcrystalline Cellulose, NF (Avicel | 10.660 | 82.00 |
| PH 102) | | |
| Colloidal Silicon Dioxide, NF | 0.0975 | 0.75 |
| Pregelatinized Corn Starch, NF | 6.435 | 49.50 |
| Croscarmellose Sodium, NF | 1.950 | 15.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water, USP* | 17.550 | 135.00 |
| Film Coating (Inactive Ingredients)** | | |
| Acryl-Eze (93018359) | 12.900 | — |
| Silicone Emulsion, 30% | 0.323 | — |
| Purified Water, USP | 51.271 | — |

*Purified water is removed during processing; mg per tablet is based on a theoretical specific gravity of 1.0 g/ml
**6 kg excess prepared to account for losses during transfer

The formulation of Example IIIB was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the croscarmellose sodium and MCC to the V-Blender and blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets.
13. After compression, spray coat the compressed dosage forms with the film coating.

### Example IV

Dissolution tests of Modified Release and Immediate Release Formulations prepared in accordance with Examples II and IIIA respectively were performed under USP 27 Dissolution Apparatus Type II Paddle Method @ 50 RPM in 900 ml water at 37±0.5° C. The delayed release formulation of Example IIIB was dissolution tested under USP 27 Dissolution Apparatus Type II Paddle Method @ 50 RPM and 37±0.5° C. for 120 minutes in acid medium (1000 ml of 0.1N hydrochloric acid), subsequently followed by buffer medium (1000 ml of pH 6.8 phosphate buffer) for 45 minutes.

The results are listed in tables 5-7 below.

TABLE 5
Dissolution Results for the Immediate Release Formulation

| Time (min.) | Dissolution (%) | Standard Deviation |
|---|---|---|
| 15 | 58.0% | ±9.521905 |
| 30 | 96.0% | ±10.2697 |
| 45 | 102.0% | ±0.408248 |
| 60 | 104.0% | ±1.032796 |

TABLE 6
Dissolution Results for the Modified Release Formulation

| Time (min.) | Dissolution (%) | Standard Deviation |
|---|---|---|
| 15 | 21.0% | ±1.414214 |
| 30 | 40.0% | ±2.810694 |
| 45 | 58.0% | ±3.600926 |
| 60 | 73.0% | ±3.81663 |
| 90 | 98.0% | ±2.097618 |

TABLE 7
Dissolution Results for the Delayed
Release Formulation (in base stage)

| Time (min.) | Dissolution (%) | Standard Deviation |
|---|---|---|
| 15 | 16% | ±6.013873 |
| 30 | 89% | ±14.06769 |
| 45 | 95% | ±2.810694 |
| 60 | 97% | ±2.345208 |

### Example V

A study assessed the bioequivalence of three new tablet formulations of tranexamic acid (650-mg modified-release (referred to as “modified immediate release”; Example I) and 650-mg delayed release (Example IIIB) in comparison with a 650-mg immediate-release formulation of the invention (Example IIIA), and to determine their oral bioavailability in comparison with intravenous administration. The study was a randomized, single-dose, 4-way crossover trial performed on 28 healthy female volunteers under fasting conditions. A total of 26 subjects completed the clinical phase of the study. The tranexamic acid doses (1.3 g for oral formulations and 1 g for intravenous formulation) were separated by a washout period of 7 days. The results indicated that the modified-release tablet was bioequivalent to the reference immediate release tablet by standard criteria. The absolute bioavailabilities were 44.9% and 46.0% respectively. The two formulations also gave similar values for mean Tₘₐₓ (2.9 and 2.8 h) and Cₘₐₓ (11.3 and 12.3 μg/mL). In contrast, the delayed-release formulation was not bioequivalent (absolute bioavailability=32.4%). For the intravenous dose, mean terminal elimination half-life was 10.2 h and plasma clearance was 8.24 L/h.

In summary, studies of the tranexamic acid modified-release tablet formulation have shown bioequivalence with the immediate-release tablet formulation, with absolute bioavailability of 45%.

### Example VI

### Menorraghia Impact Measure Validation

Objective measurements of menstrual blood loss are not practical in the healthcare setting, and they correlate poorly with a woman's subjective assessment of blood loss and its impact on quality of life [Warner 2004; National Collaborating Centre for Women's and Children's Health, 2007]. Menorrhagia is a subjective condition and may be practically defined as menstrual loss that is greater than the woman feels that she can reasonably manage. The amelioration of symptoms of heavy menstrual loss are practical efficacy benefits of the treatment are therefore important to measure and validate in a controlled clinical environment.

The MI was evaluated in a sub population of patients enrolled in a clinical trial designed to assess the safety and efficacy of tranexamic acid at an oral dose of 3.9 g administered daily for up to 5 days during each menstrual period. Two groups of patients were used to assess the MI, one group of patients were those diagnosed with menorrhagia and undergoing treatment. The second group was an age matched normal group. The sub-study was designed: to collect sufficient quantitative data to support the construct-related validation of the MI measures; to collect sufficient quantitative data to support the assessment of meaningful/important change in blood loss to the women; to conduct a test/retest evaluation of the instrument, and to address the reliability of the MI measures.

### Study Methods

Development of the MI began with a review of the literature focusing on the methods used to collect qualitative data from menorrhagia patients. Qualitative interviews with patients determined which symptomatic concepts were most important to women and could be included in a draft Impact Measure. Cognitive debriefing interviews to evaluate patient understanding of items led to the synthesis of a patient-based instrument for assessing the impact of limitations caused by heavy menstrual bleeding. Published measures were used in the evaluation of the psychometric properties of the Menorrhagia Instrument to assess Construct-Related Validity. The reference measures include, the Ruta Menorrhagia Questionnaire [Ruta 1995] and the Medical Outcomes Study Short-Form 36 Item Health Status Instrument (SF-36) [Ware 1992]. Scoring of the standardized measures followed published algorithms, Table 8.

TABLE 8
Descriptions of Instruments used in this study

| Measure | Score Generated | Score Ranges |
|---|---|---|
| Menorrhagia Impact | Blood Loss Severity (Q1) | 1 (light) thru 4 (very heavy) |
| Measure (MI) | Limitation | |
| | Work outside or inside the home (Q2) | 1 (not at all) thru 5 (extremely) |
| | Physical activities (Q3) | 1 (not at all) thru 5 (extremely) |
| | Social or leisure activities (Q4) | 1 (not at all) thru 5 (extremely) |
| | Activity list (Q5) | [Descriptive] |
| | Change in blood loss (follow-up) (Q6, 6a, 6b) | [15-pt scale: 0 = no change, |
| | Meaningful/important change (Q6c) | 1-7 improve, 1-7 worse] |
| | | Y/N |
| Ruta Menorrhagia | Global | 0 (asymptomatic)-42 (severe) |
| Questionnaire | Specific | |
| | Physical Function: Impact on work and daily | 0 (asymptomatic)-6 (severe) |
| | activities (Q9 and Q10) | |
| | Social Function: Impact on social and leisure | 0 (asymptomatic)-8 (severe) |
| | activities and sex-life (Q11 and Q12) | |
| SF-36 | Physical Functioning, Role-Physical, Bodily Pain | 0-100 |
| | General Health (can be combined to form Physical | (100 = minimal impairment) |
| | Health Component Score); Vitality, Social Functioning, | |
| | Role-Emotional, Mental Health (can be combined to | |
| | form Mental Health Component Score) | |

### Study Design

A total of 262 women completed the MI. The MI measures 1 through 5 were administered after subject's baseline period and after the subsequent first, second, third and sixth treatment periods. The MI measure 6 was administered after the first treatment period only. For this validation study, only the data collected through Month 1 of treatment was included in the analyses for the treatment cohort. The MI measures 1-5 were administered at baseline and at the subsequent first and second non-treatment periods for the subjects in the normal cohort The MI measure 6 was administered and data collected, at Month 1 and Month 2. The Ruta Menorrhagia Questionnaire, SF-36 Health Survey and the MI were completed by the subject before visit procedures were performed. A subset of at least 50 subjects were asked to return to the study site 7 to 10 days after the baseline Visit but before the next menstrual period starts to complete the MI a second time.

### Treatment Group

A total of 177 patients were enrolled into the sub-study. During this time period 28 patients withdrew consent, dropped-out, or did not properly complete MI and were non-evaluable. The 149 patients remaining were intended to be age matched. The majority of patients in the study were in their late 30's or early 40's. Because of the difficulty of enrolling sufficient numbers of women with normal menstrual periods in this age bracket 18 evaluable patients were not age matched. A total of 131 evaluable patients were age matched. A sub-set of 80 evaluable patients participated in the test/retest segment of the validation. Of these patients 11 were evaluable but not age matched. Data from all 80 patients were used for statistical evaluation of the test/re-test correlations.

### Normal Group

A group of women with self reported normal menstrual bleeding comprised the pool of normal women eligible for age matching in the study. A normal was defined as all of the following: a menstrual cycle between 26 and 32 days long, and their last (most recently completed) menstrual period was seven days or less in duration, the heaviest bleeding was three days or less, and the woman classified the bleeding overall as “light” or “moderate” as opposed to “heavy” or “very heavy. Women with normal periods who were enrolled into the study served as age-match controls for women recruited into the treatment group. Un-matching and re-matching occurred throughout the enrollment period if participants in either group dropped out of the study, if better re-matching increased the total number of matched pairs, or if the age-matched woman with normal periods did not enroll in the study.

Five women enrolled in the study did not complete the study through Visit 3. Another five women who did complete the study became ‘unmatched’ as the Treatment Group participant they had been matched to became non-evaluable. The 131 women who completed the study and remained matched are the Validation Sample Normal Group. A total of 51 women completed the Retest.

The following Measures were summarized and statistically analyzed:
MI measure1—Blood Loss Rating
MI measure 2—Limitation of Work Outside or Inside the Home
MI measure 3—Limitation of Physical Activities
MI measure 4—Limitation of Social or Leisure Activities
MI measure 6/6a/6b—Menstrual Blood Loss During Last Period
MI measure 6c—Meaningfulness of Change in Menstrual Blood Loss

The statistics include the counts (missing data), mean, standard deviation, median, inter-quartile range, and minimum/maximum values. Differences in these variables between the treatment and normal cohorts were assessed using analysis of variance.

A p-value <0.05 was required for significance using two-sided hypothesis tests; no p-value adjustments were made for the analysis of multiple endpoints. All analyses were performed under SPSS version 11.5 for Windows, and the Stuart-Maxwell test for homogeneity was performed using Stata version 9.0 for Windows.

Validation of the MI was conducted using standardized analytic procedures found in the FDA Draft Guidance on Patient Reported Outcomes for Use in Evaluating Medical Products for Labeling Claims and instrument review criteria developed by the Scientific Advisory Committee of the Medical Outcomes Trust.¹ ¹ Scientific Advisory Committee of the Medical Outcomes Trust. Assessing health status and quality-of-life instruments: attributes and review criteria. Qual Life Res. 2002; 11: 193-205

### Evaluation of the Menorrhagia Instrument

The MI consisted of 4 individual measures (1-4) that were analyzed separately for validation. No summative scale was derived. Measure 5, served as descriptive of variables and did not undergo standard validation analyses. Measures 6, 6a and 6b dealt with menstrual blood loss relative to the previous menstrual period. The answers to the measures in the subparts of measure 6, were combined to produce a 15 point rating scale. The scale values range from −7 to +7 with −7 representing a very great deal worse menstrual blood loss than the previous period, and +7 representing a very great deal better menstrual blood loss than the previous period. The midpoint (0) represents the perception of about the same menstrual blood loss as the previous period.

Test-retest reliability assessed if items produced stable, reliable scores under similar conditions (Guttman, 1945). Reproducibility was evaluated in a subset of at least 50 from the treatment group and at least 50 from the normal group 7 to 10 days after the baseline visit using the intra-class correlation coefficient (ICC, see formula below). Values above 0.70 indicated the stability of an instrument over time. The following formula was used to compute the Intraclass Correlation Coefficient (ICC):

$$ICC = \frac{{A}^{2} + {B}^{2} + {C}^{2}}{{A}^{2} + {B}^{2} + {D}^{2} - ( \frac{{C}^{2}}{n} )}$$

$$where \text{:}$$

$$A = Standard deviation of baseline score$$

$$B = Standard deviation of Time 2 score$$

$$C = Standard deviation of change in score$$

$$D = mean of change in score$$

$$n = number of respondents$$

The data for each of the measures was above 0.70. In the test population, n=88, values of 0.72(0.60-0.81), 0.75(0.64-0.83), 0.77(0.67-0.84) and 0.76(0.66-0.84) for measures 1 to 4 respectively. The aged matched normal values where n=51 were 0.77(0.63-0.86), 0.67(0.49-0.80), 0.75(0.60-0.85) and 0.86(0.77-0.92) respectively.

Construct-Related Validity was established when relationships among items, domains, and concepts conform to what was predicted by the conceptual framework for the instrument. This includes convergent, discriminant, and known-groups validity. Convergent and discriminant validity was present where measures of the same construct are more highly related and measures of different constructs were less related. To assess convergent and discriminant validity, Pearson's correlation coefficients were computed between each MI measure and items and scales from the SF-36 and the Ruta Menorrhagia Questionnaire included in the study design and administered at the same visit. The following hypotheses were tested:

The MI Blood Loss Measure (#1) will have a stronger association with the Ruta Menorrhagia Questionnaire (RMQ) than to the SF-36 subscales.

The MI Physical Activity Limitation Measure (#3) will have a stronger association with the RMQ Physical Function scale, the SF-36 Physical domain, the SF-36 Role-Physical domain, and SF-36 Physical Component Summary score than the Ruta Social, SF-36 Social, and SF-36 Vitality domains.

The MI Social/Leisure Activity Limitation will have a have stronger associations with the RMQ Social Function scale and the SF-36 Social Function domain than the RMQ Physical, the SF-36 Physical and SF-36 Bodily Pain domains.

For convergent validity, the correlations of MI measures with Ruta subscales, SF-36 subscales, and diary data are shown in Table 9. The Ruta global score was highly correlated with each MI measures (range 0.757-0.809). The correlations of items with the SF-36 subscales were low to moderate, which is to be expected since the SF-36 is not a disease-specific measure, but rather a more generic health status measure unable to detect differences between a normal population and a population of women with menorrhagia. The MI measures were more strongly correlated with the SF-36 Physical and Role Physical subscales than other SF-36 subscales.

TABLE 9
Correlations Between Menorrhagia Instrument Patient
Reported Outcome (PRO) Measures and Ruta/SF-36/Diary

| | | MI measure 2 | MI measure 3 | MI measure 4 |
|---|---|---|---|---|
| | MI measure 1 Blood | Limit work outside | Limit physical | Limit social or |
| | Loss | or inside home | activity | leisure activity |
| Ruta - | 0.767 (0.000) | 0.785 (0.000) | 0.807 (0.000) | 0.809 (0.000) |
| Global | | | | |
| Ruta - | 0.512 (0.000) | 0.682 (0.000) | 0.646 (0.000) | 0.664 (0.000) |
| Physical | | | | |
| Fx | | | | |
| Ruta - | 0.606 (0.000) | 0.634 (0.000) | 0.659 (0.000) | 0.683 (0.000) |
| Social Fx | | | | |
| SF-36 - | −0.229 (0.000) | −0.234 (0.000) | −0.264 (0.000) | −0.273 (0.000) |
| Physical | | | | |
| Fx | | | | |
| SF-36 - | −0.118 (0.057) | −0.194 (0.002) | −0.200 (0.001) | −0.261 (0.000) |
| Social Fx | | | | |
| SF-36 - | −0.200 (0.001) | −0.279 (0.000) | −0.258 (0.000) | −0.303 (0.000) |
| Role | | | | |
| Physical | | | | |
| SF-36 - | −0.143 (0.021) | −0.193 (0.002) | −0.248 (0.000) | −0.250 (0.000) |
| Vitality | | | | |
| SF-36 - | −0.087 (0.163) | −0.168 (0.006) | −0.192 (0.002) | −0.205 (0.001) |
| Bodily | | | | |
| Pain | | | | |
| SF-36 - PCS | −0.190 (0.002) | −0.271 (0.000) | −0.285 (0.000) | −0.275 (0.000) |

The data supported the hypothesis that the MI Blood Loss measure (#1) had a stronger association with the Ruta global score than to the SF-36 subscales. While the hypothesis that MI measure #3 (Physical Activity Limitation) would be strongly associated to the physical domains of the RMQ (r=0.65) and SF-36 (r=−0.26) was confirmed, this measure was also strongly correlated to the RMQ Social Functioning (r=0.66). MI measure #4 (Social or Leisure Activity Limitation) was highly correlated to the RMQ Social (r=0.68) and moderately associated with the SF-36 Social Functioning domain.

Known-groups validity determined the ability of the instrument to discriminate between groups of subjects known to be distinct. The ability of the MI items to discriminate among known groups was assessed by comparing the 4 items (1 thru 4) to responses from the two groups (treatment and normal) at baseline. Differences in these variables, between the treatment and normal groups, were assessed using analysis of variance. A p-value <0.05 was required for significance using two-sided hypothesis tests; no p-value adjustments was made for the analysis of multiple endpoints.

For each MI measure, the mean score for the treatment group was significantly different than the mean score for the normal group (p<0.001). The treatment group scores were higher for each individual measure, indicating greater limitation as a result of their excessive menstrual blood loss (see Table 10).

TABLE 10
Known-Groups Validity of the MI

| | | | | | AGE MATCH | | | |
|---|---|---|---|---|---|---|---|---|
| | | Treatment | | | NORMAL | | | |
| | | Cohort | | | Cohort | | | |
| | | | | St. | | | St. | |
| | | N | Mean | Dev. | N | Mean | Dev. | F (sig.)¹ |
| MI | Self-perceived blood loss | 131 | 3.25 | 0.61 | 131 | 2.10 | 0.61 | 234.727 |
| measure 1 | | | | | | | | (<0.001) |
| MI | Limit you in your work | 131 | 3.04 | 0.99 | 131 | 1.34 | 0.59 | 286.864 |
| measure 2 | | | | | | | | (<0.001) |
| MI | Limit you in your physical | 131 | 3.28 | 0.95 | 131 | 1.49 | 0.72 | 299.011 |
| measure 3 | activities | | | | | | | (<0.001) |
| MI | Limit you in your social/leisure | 131 | 3.05 | 1.06 | 131 | 1.37 | 0.72 | 227.312 |
| measure 4 | activities | | | | | | | (<0.001) |

The ability to detect change required that values for the item or instrument change when the concept it measures changed. In order to measure the MI items ability to detect change, longitudinal data were evaluated focusing primarily on the changes from baseline to month 1. Differences in proportions and comparisons between treatment and normal groups were compared using chi-square statistics (the Stuart-Maxwell test testing marginal homogeneity for all categories simultaneously). Cohen Effect Size statistics were also compared between the treatment and normal groups. The Cohen Effect Size was computed by taking the mean change in measure score (baseline to month 1) and dividing that by the standard deviation of mean baseline score.² ² Cohen, J. J. (1988). Statistical power analysis for the behavioral sciences (p. 8). Erlbaum: Hillsdale, N.J.

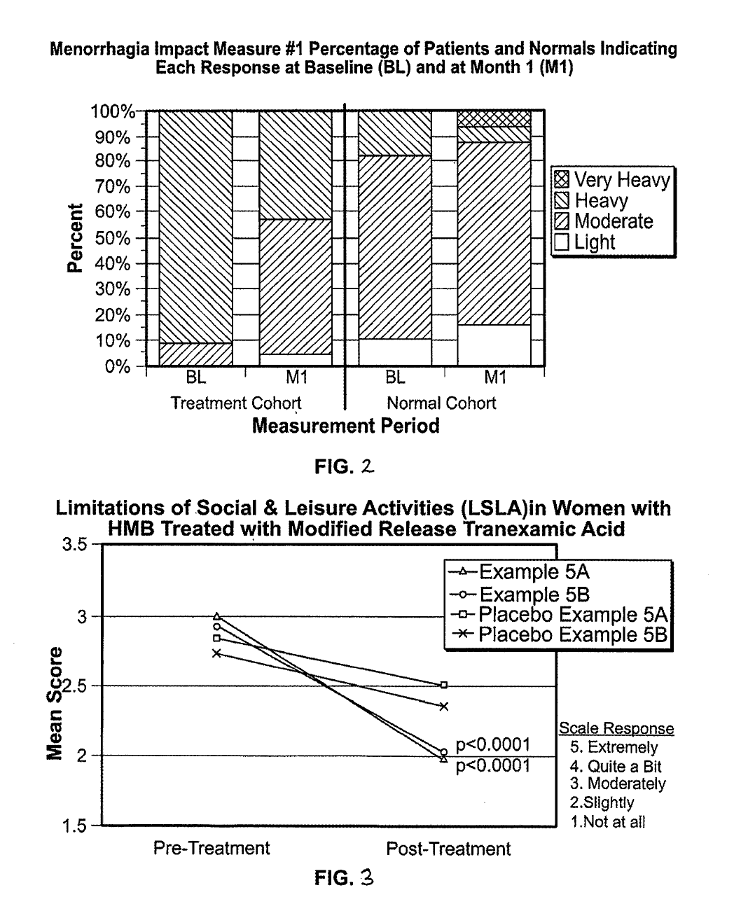

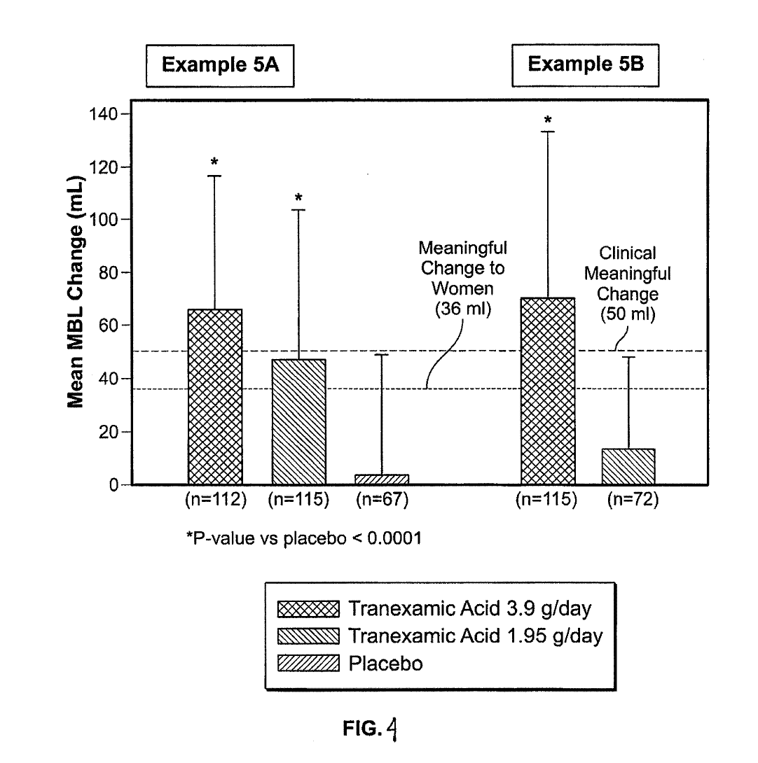

Ability to detect change was described for each item in Tables 11A-D by indicating the distribution of baseline and month 1 response option pairs for all patients. Change in responses from baseline to month 1 was tested using the Stuart-Maxwell test. For the treatment group, there was significant change in responses to each measure from baseline to month one (p<0.001). For the normal group, none of the items had significant changes in responses from baseline to month one. FIG. 2 illustrates the distribution of responses to measure 1 at baseline and at month one. In the treatment group, the proportion reporting light or moderate bleeding as measured with item 1, increased from baseline to month 1, and in the normal group this proportion changed very little.

TABLE 11A
Sensitivity to change of the MI Measure 1

| | | | Month 1 | | | | |
|---|---|---|---|---|---|---|---|
| | | Response | | | | Very | Stuart-Maxwell test of |
| Cohort | | category | Light | Moderate | Heavy | Heavy | association |
| Treatment | Baseline | Light | 0 (0.0%) | 0 (0.0%) | 0 (0.0%) | 0 (0.0%) | 59.09 (p < 0.001) |
| | | Moderate | 0 (0.0%) | 8 (6.3%) | 4 (3.2%) | 0 (0.0%) | |
| | | Heavy | 3 (2.4%) | 41 (32.5%) | 24 (19.0%) | 2 (1.6%) | |
| | | Very Heavy | 2 (1.6%) | 18 (14.3%) | 13 (10.3%) | 11 (8.7%) | |
| Normal | Baseline | Light | 9 (6.9%) | 5 (3.8%) | 0 (0.0%) | 0 (0.0%) | 6.35 (p = 0.130) |
| | | Moderate | 12 (9.2%) | 77 (59.2%) | 4 (3.1%) | 0 (0.0%) | |
| | | Heavy | 0 (0.0%) | 9 (6.9%) | 8 (6.2%) | 2 (1.5%) | |
| | | Very Heavy | 0 (0.0%) | 2 (1.5%) | 2 (1.5%) | 0 (0.0%) | |

TABLE 11B
Sensitivity to change of the MI Measure 2

| | | | Month 1 | | | | | |
|---|---|---|---|---|---|---|---|---|
| | | | | | | | | Stuart-Maxwell |
| | | Response | Not at | | | Quite a | | test of |
| Cohort | | category | all | Slightly | Moderately | bit | Extremely | association |
| Treatment | Baseline | Not at all | 5 (4.0%) | 0 (0.0%) | 1 (0.8%) | 1 (0.8%) | 0 (0.0%) | 53.33 (p < 0.001) |
| | | Slightly | 12 (9.5%) | 11 (8.7%) | 2 (1.6%) | 1 (0.8%) | 0 (0.0%) | |
| | | Moderately | 17 (13.5%) | 26 (20.6%) | 14 (11.1%) | 1 (0.8%) | 0 (0.0%) | |
| | | Quite a bit | 2 (1.6%) | 8 (6.3%) | 5 (4.0%) | 9 (7.1%) | 0 (0.0%) | |
| | | Extremely | 3 (2.4%) | 3 (2.4%) | 3 (2.4%) | 1 (0.8%) | 1 (0.8%) | |
| Normal | Baseline | Not at all | 89 (69.0%) | 5 (3.9%) | 1 (0.8%) | 0 (0.0%) | 0 (0.0%) | 2.86 (p = 0.517) |
| | | Slightly | 8 (6.2%) | 13 (10.1%) | 4 (3.1%) | 2 (1.6%) | 0 (0.0%) | |
| | | Moderately | 0 (0.0%) | 3 (2.3%) | 4 (3.1%) | 0 (0.0%) | 0 (0.0%) | |
| | | Quite a bit | 0 (0.0%) | 0 (0.0%) | 0 (0.0%) | 0 (0.0%) | 0 (0.0%) | |
| | | Extremely | 0 (0.0%) | 0 (0.0%) | 0 (0.0%) | 0 (0.0%) | 0 (0.0%) | |

TABLE 11C
Sensitivity to change of the MI Measure 3

| | | | | | | | | Stuart- |
|---|---|---|---|---|---|---|---|---|
| | | | Month 1 | | | | | Maxwell test |
| | | Response | Not at | | | Quite a | | of |
| Cohort | | category | all | Slightly | Moderately | bit | Extremely | association |
| Treatment | Baseline | Not at all | 0 (0.0%) | 0 (0.0%) | 1 (0.8%) | 0 (0.0%) | 0 (0.0%) | 64.58 (p < 0.001) |
| | | Slightly | 12 (9.5%) | 12 (9.5%) | 1 (0.8%) | 1 (0.8%) | 0 (0.0%) | |
| | | Moderately | 14 (11.1%) | 20 (15.9%) | 11 (8.7%) | 3 (2.4%) | 0 (0.0%) | |
| | | Quite a bit | 6 (4.8%) | 17 (13.5%) | 9 (7.1%) | 5 (4.0%) | 0 (0.0%) | |
| | | Extremely | 5 (4.0%) | 2 (1.6%) | 2 (1.6%) | 3 (2.4%) | 2 (1.6%) | |
| Normal | Baseline | Not at all | 72 (55.4%) | 9 (6.9%) | 0 (0.0%) | 0 (0.0%) | 0 (0.0%) | 1.99 (p = 0.708) |
| | | Slightly | 14 (10.8%) | 18 (13.8%) | 3 (2.3%) | 1 (0.8%) | 0 (0.0%) | |
| | | Moderately | 0 (0.0%) | 6 (4.6%) | 4 (3.1%) | 1 (0.8%) | 0 (0.0%) | |
| | | Quite a bit | 0 (0.0%) | 1 (0.8%) | 1 (0.8%) | 0 (0.0%) | 0 (0.0%) | |
| | | Extremely | 0 (0.0%) | 0 (0.0%) | 0 (0.0%) | 0 (0.0%) | 0 (0.0%) | |

TABLE 11D
Sensitivity to change of the MI Measure 4

| | | | | | | | | Stuart- |
|---|---|---|---|---|---|---|---|---|
| | | | Month 1 | | | | | Maxwell test |
| | | Response | Not at | | | Quite a | | of |
| Cohort | | category | all | Slightly | Moderately | bit | Extremely | association |
| Treatment | Baseline | Not at all | 6 (4.8%) | 3 (2.4%) | 0 (0.0%) | 0 (0.0%) | 0 (0.0%) | 60.77 (p < 0.001) |
| | | Slightly | 16 (12.7%) | 10 (7.9%) | 0 (0.0%) | 2 (1.6%) | 0 (0.0%) | |
| | | Moderately | 19 (15.1%) | 14 (11.1%) | 12 (9.5%) | 2 (1.6%) | 1 (0.8%) | |
| | | Quite a bit | 5 (4.0%) | 14 (11.1%) | 4 (3.2%) | 6 (4.8%) | 0 (0.0%) | |
| | | Extremely | 3 (2.4%) | 4 (3.2%) | 1 (0.8%) | 3 (2.4%) | 1 (0.8%) | |
| Normal | Baseline | Not at all | 84 (64.6%) | 11 (8.5%) | 0 (0.0%) | 0 (0.0%) | 0 (0.0%) | 1.71 (p = 0.807) |
| | | Slightly | 10 (7.7%) | 14 (10.8%) | 2 (1.5%) | 0 (0.0%) | 0 (0.0%) | |
| | | Moderately | 0 (0.0%) | 4 (3.1%) | 2 (1.5%) | 0 (0.0%) | 0 (0.0%) | |
| | | Quite a bit | 0 (0.0%) | 0 (0.0%) | 0 (0.0%) | 2 (1.5%) | 0 (0.0%) | |
| | | Extremely | 1 (0.8%) | 0 (0.0%) | 0 (0.0%) | 0 (0.0%) | 0 (0.0%) | |

The amount of change in each item from baseline to month 1 is shown in Table 12. For the treatment group, the mean change in response from baseline to month 1 ranged from −0.76 to −1.16 for the four items. The calculated effect size shows this amount of change for each item ranged from −0.9 to −1.2. For the normal group, the mean change in response from baseline to month 1 ranged from 0.03 to −0.12 for the four items. The effect size for each item ranged from 0.053 to −0.197. This analysis shows a large response in patients undergoing treatment and little to no response in normal women who have received no treatment. This instrument is capable of identifying the perceived improvement in menstrual blood loss.

TABLE 12
Sensitivity to Change of MI Effect Size

| | | BASELINE | | | MONTH 1 | | | CHANGE | | | |
|---|---|---|---|---|---|---|---|---|---|---|---|
| | | | | St | | | St | | | St | Effect |
| Menorrhagia Item | | n | Mean | Dev | n | Mean | Dev | n | Mean | Dev | Size¹ |
| Item 1 | Self-perceived blood | 126 | 3.25 | 0.62 | 126 | 2.49 | 0.73 | 126 | −0.76 | 0.84 | −1.226 |
| | loss | | | | | | | | | | |
| Item 2 | Limit you in your | 126 | 3.05 | 0.99 | 126 | 2.12 | 0.99 | 126 | −0.93 | 1.13 | −0.939 |
| | work | | | | | | | | | | |
| Item 3 | Limit you in your | 126 | 3.29 | 0.95 | 126 | 2.13 | 1.00 | 126 | −1.16 | 1.17 | −1.221 |
| | physical activities | | | | | | | | | | |
| Item 4 | Limit you in your | 126 | 3.06 | 1.06 | 126 | 2.00 | 1.04 | 126 | −1.06 | 1.19 | −1.000 |
| | social/leisure | | | | | | | | | | |
| | activities | | | | | | | | | | |

| | | BASELINE | | | | | | | | |
|---|---|---|---|---|---|---|---|---|---|---|
| | | | | St | CHANGE | | | | St | Effect |
| Menorrhagia Item | | n | Mean | Dev | n | Mean | n | Mean | Dev | Size |
| Item 1 | Self-perceived blood | 130 | 2.10 | 0.61 | 130 | 1.98 | 130 | −0.12 | 0.56 | −0.197 |
| | loss | | | | | | | | | |
| Item 2 | Limit you in your | 129 | 1.32 | 0.57 | 129 | 1.35 | 129 | 0.03 | 0.50 | 0.053 |
| | work | | | | | | | | | |
| Item 3 | Limit you in your | 130 | 1.49 | 0.72 | 130 | 1.43 | 130 | −0.06 | 0.57 | −0.083 |
| | physical activities | | | | | | | | | |
| Item 4 | Limit you in your | 130 | 1.37 | 0.72 | 130 | 1.33 | 130 | −0.04 | 0.58 | −0.056 |
| | social/leisure | | | | | | | | | |
| | activities | | | | | | | | | |

Responses from treatment group participants were divided based on two separate responder definitions. In the first definition, a responder was a patient indicating a one-category change in MI measure 1. In the second definition, a responder was a patient who entered the study as “Very heavy” or “Heavy” (MI measure 1) and then, following treatment (month 1), indicated being “Moderate” or “Light”. When the treatment group was analyzed using the first responder definition, 69 (90%) of the 77 responders reported improvement and 63 (91%) of these rated this improvement as “a meaningful change”. Thirty-five (71%) of the 49 non-responders reported improvement and 35 (92%) rated their change as “a meaningful change”.

When the treatment group was analyzed using the second responder definition, 57 (89%) of the 64 responders reported improvement, and 52 (91%) reported their change to be meaningful. Forty-seven (76%) of the 62 non-responders reported improvement, and 45 (90%) reported their change to be meaningful. Among the normal group, 96 (73%) of 130 patients reported no change. Twenty-one (16%) reported improvement, and 13 (10%) reported worsening. Of the patients reporting change, 15 (44%) rated the change as “a meaningful change”.

For those women on treatment who reported a meaningful improvement (78.6%), MI items 3 and 4 showed the greatest treatment effect with improvements of 1.29 and 1.17, respectively. As expected, the majority of the Normal cohort (73.3%) reported no change in their menstrual period.

### Example VII

The following clinical study was carried out in order to evaluate the efficacy and safety of tranexamic acid provided as an oral modified release formulation of Example II to reduce menstrual blood loss (MBL) in women with menorrhagia when administered during menstruation compared to placebo.

This was a multi-center, double-blind, placebo-controlled, parallel-group study. The study consisted of a screening phase of two (2) menstrual periods (no treatment) to determine eligibility, followed by a treatment phase spanning three (3) menstrual periods to assess the efficacy and safety of tranexamic acid during menstruation.

The primary objective of the study was to determine the efficacy of a 1.95 gm/day of tranexamic acid (650 mg orally three times daily, TID) and 3.9 gm/day of tranexamic acid (1.3 gm orally three times daily, TID) administered during menstruation for up to 5 days (maximum of 15 doses) to reduce menstrual blood loss in women with objective evidence of heavy menstrual bleeding.

The secondary objective of the study was to determine the improvement with administration of 1.95 gm/day or 3.9 gm/day of tranexamic acid in women with heavy menstrual bleeding in their symptoms including, Limitation in Social Leisure Activities (LSLA) and Limitation in Physical Activities (LPA) scores from the Menorrhagia Instrument Measures (FIG. 1). Further the objective was to determine the safety of the 1.95 gm/day and 3.9 gm/day of the modified release tranexamic acid formulation administered during menstruation.

Three treatment periods were averaged for the menstrual blood loss (MBL) primary efficacy evaluation (first, second, and third periods on treatment). All periods were evaluated for the secondary endpoints, and for safety of tranexamic acid at an oral dose of 1.3 gm or placebo administered three (3) times daily for up to five consecutive (5) days (maximum of 15 doses) during menstruation.

Criteria for Evaluation (Safety and Efficacy Assessments):

### Efficacy Assessment

Menstrual blood loss (MBL) was assessed during the entire menstrual period by the alkaline hematin test (AHT) method. The Menorrhagia Instrument Measures (FIG. 1) were also administered immediately after each menstrual period under investigation. For the Primary Endpoint, the objective reduction in menstrual blood loss (MBL) during the entire menstrual period as assessed by the AHT Method was assessed.

For the Secondary Endpoints, the scores for Limitation in Social Leisure Activities (LSLA) and the scores for Limitation in Physical Activities (LPA) from the Menorrhagia Instrument Measures (MI), measures #4 and #3, respectively) were assessed.

For the Secondary Endpoints the data collected included at least; Menstrual Blood Loss (MBL) assessment score (MI measure 1), Limitation in Work Outside or Inside the Home (LWH) score (MI item 2), and subject assessment of meaningfulness score from the MI (measure 6) (used for the MBL responder analysis).

### Efficacy Results

The efficacy results were based on the modified ITT (mITT) populations. Results from the analysis of other populations were very similar to those derived from the analysis of the mITT population, and do not alter the general conclusions presented below. The numbers of subjects in the mITT populations in the efficacy study are summarized in Table 13 below:

TABLE 13
Numbers of Subjects in MITT Populations
in Pivotal Efficacy Studies

| Treatment | N |
|---|---|
| Placebo | 67 |
| Tranexamic acid (1.95 g/day) | 115 |
| Tranexamic acid (3.9 g/day) | 112 |

### Primary Efficacy Endpoint

Subjects in both treatment groups experienced a significant reduction from baseline in mean MBL The mean reduction in MBL in subjects treated with the higher dose (3.9 g/day) was 65.3 mL, or 38.6% compared with the baseline value (p<0.0001). A smaller reduction was observed in subjects at the lower dose of 1.95 g/day (46.5 mL, 26.1%, p<0.0001). The reductions in both groups were statistically significant (p<0.0001) when compared with that in the placebo control group (2.98 mL).

### Key Secondary Efficacy Endpoints

Significant treatment-related reductions from baseline in mean LSLA score and mean LPA score were observed. Other secondary efficacy endpoints provided supportive evidence of the efficacy of tranexamic acid. Specifically, subjects' assessments of MBL (MI item 1) and LWH (MI measure 2), were both significantly reduced for subjects in the 3.9 g/day tranexamic acid group compared with placebo. The number of patients responding to treatment was assessed. A responder was defined as a subject with a reduction in MBL and a subjective “meaningful” improvement according to the MI (measure 6c) after the first menstrual cycle during the treatment period. The proportion of responders in this study was 58.3% and 71.0% in the 1.95 and 3.9 g/day tranexamic acid groups respectively, compared with placebo response rate of 23.4% (p<0.0001 for both dose levels).

These results demonstrate that tranexamic acid at doses of 1.9 and 3.9 g/day ameliorates the symptoms associated with HMB, including at least limitations in social, leisure, and physical functioning. In addition, these results provide converging evidence that tranexamic acid modified-release tablets are efficacious in the treatment of HMB.

### Example VIII

The following clinical study was carried out in order to evaluate the efficacy and safety of the modified release (MR) oral formulation of tranexamic acid of Example Ito reduce menstrual blood loss (MBL) in women with menorrhagia when administered during menstruation compared to placebo.

This was a multi-center, double-blind, placebo-controlled, parallel-group study. The study consisted of a screening phase of two (2) menstrual periods (no treatment) to determine eligibility, followed by a treatment phase spanning six (6) menstrual periods to assess the efficacy and safety of tranexamic acid during menstruation.

The primary objective of the study was to determine the efficacy of a 3.9 gm/day (1.3 gm orally three times daily, TID) administered during menstruation for up to 5 days (maximum of 15 doses) to reduce menstrual blood loss in women with objective evidence of heavy menstrual bleeding.

The secondary objective of the study included an evaluation of the improvement observed from 3.9 gm/day of the modified release tranexamic acid formulation administered during menstruation in women with heavy menstrual bleeding on Limitation in Social Leisure Activities (LSLA)(item 4) and Limitation in Physical Activities (LPA) (MI measure #3) scores from the Menorrhagia Instruments (FIG. 1). Four treatment periods were averaged for the menstrual blood loss (MBL) primary efficacy evaluation (first, second, third and sixth periods). All periods were evaluated for the secondary endpoints, the secondary endpoints, and for safety of tranexamic acid at an oral dose of 1.3 gm or placebo administered three (3) times daily for up to five consecutive (5) days (maximum of 15 doses) during menstruation.

### Criteria for Evaluation

Menstrual blood loss (MBL) was assessed during the entire menstrual period by the alkaline hematin test (AHT) method. Measures from the Menorrhagia Instrument (FIG. 1) were also administered immediately after each menstrual period under investigation. Subjects reported large stains exceeding the capacity of sanitary protection (and other patient reported outcome [PRO] items) during the menstrual period in daily diaries.

For the Primary Endpoint, the objective reduction in menstrual blood loss (MBL) during the entire menstrual period as assessed by the AHT Method was assessed.

For the Secondary Endpoints, the Limitation in Social Leisure Activities (LSLA) and the Limitation in Physical Activities (LPA) scores from the Menorrhagia Instrument (MI measures #4 and #3, respectively) and the total number of large stains responder analysis during the menstrual period from subject diaries were assessed.

For the Secondary Endpoints, assessment of the following were included, Menstrual Blood Loss (MBL) assessment score (MI measure #1), Limitation in Work Outside or Inside the Home (LWH) score (MI measure #2), and subject assessment of meaningfulness score from the MI (Measure #6) (used for the MBL responder analysis).

### Efficacy Results

The efficacy results were based on the modified ITT (mITT) populations. The numbers of subjects in the mITT populations in the efficacy study are summarized in the Table below:

TABLE 14
Numbers of Subjects in MITT Populations
in Pivotal Efficacy Studies

| Treatment | N |
|---|---|
| Placebo | 72 |
| Tranexamic acid (3.9 g/day) | 115 |

### Primary Efficacy Endpoint

Subjects experienced a significant reduction from baseline in mean MBL. The mean reduction in MBL in the tranexamic acid-treated subjects was 69.6 mL, or 40.4% compared with the baseline value (p<0.0001). The reduction in MBL was also statistically significant (p<0.0001) when compared with that in the placebo control group (12.6 mL, 8.2%).

### Secondary Efficacy Endpoints

For the secondary efficacy endpoints, significant treatment-related reductions from baseline in mean LSLA score and mean LPA score were observed. Subjects' assessments of MBL (MI measure #1) and LWH (MI measure #2), were both significantly reduced for subjects in the 3.9 g/day tranexamic acid group compared with placebo.

The number of patients responding to treatment was assessed as described in the previous example. A responder was defined as a subject with a reduction in MBL and a subjective “meaningful” improvement according to the MI (measure #6c) after the first menstrual cycle during the treatment period. The proportion of responders increased in the 3.9 g/day tranexamic acid treatment group (65.4%) compared with the placebo group (31.8%, p<0.0001). These results demonstrate that 3.9 g/day tranexamic acid ameliorates the symptoms associated with HMB, including improvement in limitations in social, leisure, and physical functioning. In addition, these results provide converging evidence that tranexamic acid modified-release tablets are efficacious in the treatment of HMB.

In both the Example VII and Example VIII studies, the reduction in menstrual blood loss (MBL) was evident in the first menstrual period after commencing treatment with 3.9 g/day tranexamic acid. The response to treatment was maintained for the duration of the study (three and six menstrual cycles in Example VII and Example VIII respectively; Regression analysis in the study of Example VIII confirmed that the response to tranexamic acid was durable over the six menstrual cycles (regression slope of −0.90 mL/cycle, p=0.615).

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

What is claimed is:

1. A method of using tranexamic acid in the treatment of heavy menstrual bleeding for a female patient in need thereof comprising:
a) determining the perceived amount of blood loss during a patient's most recent menstrual period, wherein the perceived amount of blood loss is determined based upon the patient's response about their blood loss on a four point scale; said scale comprising a response selected from the group consisting of i) “light”, ii) “moderate”, iii) “heavy”, and iv) “very heavy”;
b) identifying a patient in need of therapy as one with a perceived blood loss of greater than or equal to a response of “heavy”;
c) administering a therapeutically effective treatment regimen of a modified release tranexamic acid formulation for treating heavy menstrual bleeding, said treatment regimen to be administered beginning at the start of the patient's next menstrual period for at least about 3 days;
d) determining a change in the perceived amount of blood loss after administration of the tranexamic acid treatment regimen, and determining whether that change was meaningful to the patient, wherein the change in the perceived amount of blood loss is determined based upon the patient's response on a three point scale comprising a response selected from the group consisting of i) “about the same”, ii) “better”, and iii) “worse”, and the meaningfulness of the change to the patient is based upon the patient's response about the change on a two point scale comprising a response selected from the group consisting of “yes” and “no”; and
e) classifying the patient as a responder when the patient's response to the change in perceived amount of blood loss in step (d) is “better”, and the patient perceives the change to be meaningful.

2. The method of claim 1, wherein step a) further comprises determining the perceived work limitation during a patient's most recent menstrual period, wherein the perceived work limitation is determined based upon the patient's response about how much their blood loss limited their work outside or inside the home, the patient's response being based on a five point scale selected from the group consisting of i) “not at all”, ii) “slightly”, iii) “moderately”, iv) “quite a bit”, and v) “extremely”.

3. The method of claim 2, wherein step a) further comprises determining the perceived limitation on the patient's physical activities during a patient's most recent menstrual period, wherein the perceived limitation is determined based upon the patient's response about how much their blood loss limited their physical activities, the patient's response being based on a five point scale selected from the group consisting i) “not at all”, ii) “slightly”, iii) “moderately”, iv) “quite a bit”, and v) “extremely”.

4. The method of claim 3, wherein step a) further comprises determining the perceived limitation on social and leisure activities during a patient's most recent menstrual period, wherein the perceived limitation is determined based upon the patient's response about how much their blood loss limited their social and leisure activities, the patient's response being based on a five point scale selected from the group consisting of i) “not at all”, ii) “slightly”, iii) “moderately”, iv) “quite a bit”, and v) “extremely”.

5. The method of claim 4, wherein step a) further comprises determining the specific activities that were limited by the patient's blood loss during a patient's most recent menstrual period, wherein the specific activities are selected from the group consisting of walking, standing, climbing stairs, squatting or bending down, childcare, shopping, home management, leisure, exercise, sports, gardening, traveling or vacation and any other physical, social or leisure activity.

6. The method of claim 2, further comprising classifying the patient as a responder to the administration of the tranexamic acid treatment regimen based upon the patient's response to an inquiry about the perceived work limitation, wherein the patient experiences an improvement in the perceived limitation of at least one unit.

7. The method of claim 3, further comprising classifying the patient as a responder to the administration of the tranexamic acid treatment regimen based upon the patient's response to an inquiry about the perceived limitation on the patient's physical activities, wherein the patient experiences an improvement in the perceived limitation of at least one unit.

8. The method of claim 4, further comprising classifying the patient as a responder to the administration of the tranexamic acid treatment regimen based upon the patient's response to an inquiry about the perceived limitation on the patient's social and leisure activities, wherein the patient experiences an improvement in the perceived limitation of at least one unit.

9. The method of claim 6, wherein the at least one unit improvement in perceived limitation is selected from the group consisting of i) “extremely” to “quite a bit”, ii) “quite a bit” to “moderately”, iii) “moderately” to “slightly”, and iv) “slightly” to “not at all”.

10. The method of claim 7, wherein the at least one unit improvement in perceived limitation is selected from the group consisting of i) “extremely” to “quite a bit”, ii) “quite a bit” to “moderately”, iii) “moderately” to “slightly”, and iv) “slightly” to “not at all”.

11. The method of claim 8, wherein the at least one unit improvement in perceived limitation is selected from the group consisting of i) “extremely” to “quite a bit”, ii) “quite a bit” to “moderately”, iii) “moderately” to “slightly”, and iv) “slightly” to “not at all”.

12. A method of diagnosing and treating heavy menstrual bleeding comprising assessing, and monitoring the symptoms of heavy menstrual bleeding comprising assessing the patient's blood loss during their most recent menstrual period, assessing the symptomatic limitation on physical activities, assessing the limitation on social/leisure activities; identifying a patient in need of therapy as one with a perceived blood loss of greater to or equal to heavy or very heavy; administering a treatment regimen of a tranexamic acid comprising at least 1.9 g/day for at least one day of the monthly menstrual period; determining the change in perceived blood loss and symptoms; determining the change in blood loss as, about the same, better, or worse, wherein the patient perceives the change as meaningful or not meaningful; classifying the patient as a responder to the administration of tranexamic acid when the blood loss response is at least the same or better, and the patient perceives the change to be meaningful, and optionally the patient experiences at least one category change in one symptomatic measure selected from the group of social/leisure measures or physical measures.
